SEQUENCE LISTING (1 sequence)

SEQ ID NO: 1 - protein, 7 aa, Human
  ref <301>: AUTHORS: J.McLauren et. al.
  ref <302>: TITLE: Therapeutically effective antibodies against amyloid peptide target amyloid-residues 4-10
  ref <303>: JOURNAL: Nature Medicine
  ref <304>: VOLUME: 8
  ref <306>: PAGES: 1263-1269
  ref <307>: DATE: 2002-11-01
  ref <313>: RELEVANT RESIDUES: (1)..(6)
PRHDSGY

(19) United States
(12) Patent Application Publication
(10) Pub. No.: US 2005/0106626 A1
(43) Pub. Date: May 19, 2005

(54) Ligand binding of amyloid peptide protein epitope

(76) Inventor: George Pieczenik, New York, NY (US)

Correspondence Address: DR. GEORGE PIECZENIK, APT. 1F, 412 EAST 55TH STREET, NEW YORK, NY, 10022, US

(21) Appl. No.: 10/690,031

(22) Filed: Oct. 20, 2003

Related U.S. Application Data
(60) Provisional application No. 60/319,632, filed on Oct. 20, 2002.

(51) Int. Cl. G01N 33/53; C07K 7/06; C07K 5/06

(52) U.S. Cl. 435/7.1; 436/518; 530/328; 530/330; 530/329; 530/331

(57) ABSTRACT

A Ligand Product and Method for Binding Amyloid Precursor Protein as a Diagnostic and Treatment Modality for Alzheimer's Disease and Other Neurological Syndromes

### CROSS REFERENCE TO RELATED APPLICATIONS

Priority is claimed from the provisional patent 60/319,632 filed on Oct. 20, 2002 which is hereby incorporated by reference.

### BACKGROUND OF INVENTION

Elan pharmaceuticals have developed a vaccine for treatment of Alzheimer's Disease. It is based on using the Amyloid-â Peptide₄₂ as an immunigen. Recently the epitope for that immunigen has been identified as a subsection of this peptide sequence.

(Therapeutically effective antibodies against amyloid-â peptide target amyloid-residues 4-10 and inhibit cytotoxicity and fibrillogenesis J. McLaurin1, R. Cecal, M. E., Kierstead, X. Tian, A. L., Phinney, M. Manea, J. E. French, M. H. L. Lambermo, A. A. Darabie, M. E. Brown, C. Janus, M. A. Chishti, P. Horne, D. Westaway, P. E. Fraser, H. T. J. Mount, M. Przybylski & P. St George-Hyslop, Nature Medicine, published online Oct. 15, 2002).

It is sequence FRHDSGY. To be called the FRH epitope.

Methods developed by Pieczenik U.S. Pat. No. 5,866,363, U.S. Pat. No. 6,605,448 and Pieczenik Provisional patent filings are to be used to make and identify a small peptide ligand in the range of 3-13 amino acids which binds this sequence with sufficient specificity as to bind the FRH epitope.

Solid state peptide synthesis methods are well known ordinary practitioners in the art and are based on the original Merrifield synthesis methods of building peptides on resins.

The Pieczenik patents are incorporated by reference.

This method requires screening a library of combinatorial peptides either recombinantly created or chemically. The screening may also require amplification, isolation and sequencing.

A vaccine which induces antibodies to bind amyloid protein is believed to inhibit the course of Alzheimer's disease. Amyloid protein aggregates are believed to be one causal factor for Alzheimer's disease and other neurological disorders.

Previous problems with the Elan vaccine to the complete peptide and aggregates are that serious inflammation resulted and clinical trials were terminated.

This invention will allow a small ligand to act in a similar fashion without te clinical problems clinical associated with inducing an immune response and the corresponding antibody. In addition, the peptide ligand identified will allow an identification of its sequence location in the Genome data base. This allows for the identification and isolation of the authentic complete protein or similar binders to this epitope as they naturally occur.

### SUMMARY OF INVENTION

A peptide ligand isolated from a screen of peptide libraries which binds to an Amyloid Precursor Protein epitope which has been identified as being the binding site of the induced antibody.

### BRIEF DESCRIPTION OF SEQUENCES

The amino acid sequence that is called the FRH epitope is FRHDSGY.

### DETAILED DESCRIPTION

The Invention is a method to make and the product ligand which binds the peptide sequence FRHDSGY. This peptide and chemical mimic binding product is to be used to image this protein in histological stains, inter alia, as well as in NMR scans, inter alia. FITC, inter alia, and Gadolinium (Gd) and other Lanthium elements, inter alia can be attached to this binding ligand. The binding ligand can have by example DTPA or diethylenetriaminepentaacetic acid or pentetic acid as a chelating agent for the Lanthium elements. This will allow visualization by MRI and NMR. It's utility is that it can be a lead compound, a diagnostic and a treatment for Alzheimer's Disease, inter alia, and other neurological disorders. This may be a general diagnostic and treatment for all prion induced diseases.

1. The peptide sequence FRHDSGY attached to a resin and accessible to binding.

2. A peptide sequence isolated from a combinatorial library of peptides in the range of 3 to 13 amino acids which binds said bindable peptide sequence of claim 1.

3. A method of making the peptide sequence of claim 1 accessible to binding comprising the steps of chemically synthesizing said peptide sequence on a resin, and leaving said peptide sequence attached to said resin.

4. A method of binding said combinatorial library of peptides in the range of 3 to 13 amino acids to said bindable and accessible peptide sequence of claim 1.

5. The peptide sequence of claim 2 comprising imaging attachments comprising FITC, XITC, Gadolinium, Lanthium elements, diethylenetriaminepentaacetic acid, pentetic acid allowing the visualization of the binding by histological methods and MRI methods.

6. The peptide sequence of claim 2 as a diagnostic and treatment palliative for Alzheimer's Disease.
